(19) United States
(12) Patent Application Publication
(10) Pub. No.: US 2009/0054961 A1
(43) Pub. Date: Feb. 26, 2009

(54) DRUG ELUTING COATINGS FOR A MEDICAL LEAD AND METHOD

(76) Inventors: Harshad M. Borgaonkar, Blaine, MN (US); Ronald W. Heil, JR., Roseville, MN (US); Arienne P. Simon, Lino Lakes, MN (US); Jeannette C. Polkinghorne, St. Anthony, MN (US); Daniel J. Cooke, Roseville, MN (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(21) Appl. No.: 12/234,081

(22) Filed: Sep. 19, 2008

Related U.S. Application Data
(63) Continuation of application No. 11/221,588, filed on Sep. 8, 2005, now abandoned.

(51) Int. Cl. A61N 1/05

(52) U.S. Cl. 607/116

(57) ABSTRACT

A medical electrical lead includes a drug eluting coating provided over at least a portion of the lead body. The drug eluting coating can be provided over at least a portion of the lead body and adjacent to at least one electrode located on the lead body. The drug eluting coating can include at least one matrix polymer layer including a polymer admixed with a therapeutic agent. The therapeutic agent, for example, can be an anti-proliferative agent or an anti-inflammatory agent. The matrix polymer can include a medical adhesive. The rate of elution of the drug from the matrix polymer layer is affected by the drug to polymer ratio of the drug in the matrix polymer layer.

### RELATED APPLICATION

This application is a continuation-in-part of U.S. application Ser. No. 11/221,588, entitled “DRUG ELUTING COATINGS FOR A MEDICAL ELECTRICAL LEAD AND METHOD THEREFOR,” filed on Sep. 8, 2005, the entirety of which is incorporated herein by reference.

### TECHNICAL FIELD

This invention relates to the field of medical electrical leads, and more specifically to medical electrical leads including therapeutic agent eluting coatings.

### BACKGROUND

Leads having electrodes implanted in or about the heart have been used to reverse life-threatening arrhythmia or to stimulate contraction of the heart. Electrical energy is applied to the heart via an electrode to return the heart to normal rhythm. Leads are usually positioned on or in the ventricle or the atrium and the lead terminals are attached to a pacemaker or defibrillator which is implanted subcutaneously.

An issue concerning, for example, pacemaker leads is the increase in stimulation threshold, both acute and chronic, caused by the interaction between the electrode and body tissue at the point of implant. Approaches to reducing the threshold include silicone rubber based drug collars or plugs containing dexamethasone. However, in both cases, the lead design needs to accommodate the physical size of the plug or collar matrix. The size constraints imposed on the plug or collar matrix by the lead design limit the pharmacological therapy that can be provided to treat the complex nature of the natural healing process. Moreover, these devices fail to address many of the physiological processes involved in the healing response upon lead implantation. Thus, there is a need for leads and/or electrodes that are constructed to more fully address the healing process so as to maintain optimal acute and chronic thresholds.

### SUMMARY

According to various embodiments, the present invention is medical electrical lead including: a lead body having an outer surface extending from a proximal end adapted to be connected to a pulse generator to a distal end; at least one conductor operatively connected to the pulse generator extending within the lead body; and at least one electrode located on the lead body operatively connected to the at least one conductor. A drug eluting coating is disposed over at least a portion of the outer surface of the lead body adjacent to the at least one electrode. In some embodiments, the drug eluting coating includes at least one matrix polymer layer comprising a polymer admixed with a therapeutic agent including an anti-inflammatory agent. The anti-inflammatory agent can be any one of dexamethasone, dexamethasone sodium phosphate, dexamethasone acetate, betamethasone; beclomethasone, clobetasol, or mometasone furoate. In certain embodiments, the therapeutic agent includes a single anti-inflammatory agent.

According to other various embodiments, the present invention is a medical electrical lead including: a lead body having an outer surface extending from a proximal end adapted to be connected to a pulse generator to a distal end; at least one conductor operatively connected to the pulse generator extending within the lead body; and at least one electrode located on the lead body operatively connected to the at least one conductor. A drug eluting coating is disposed over at least a portion of the outer surface of the lead body adjacent to the at least one electrode. The drug eluting coating includes at least one matrix polymer layer comprising a polymer admixed with a therapeutic agent including an anti-proliferative agent. The anti-proliferative agent can be any one of paclitaxel, sirolimus, everolimus, tacrolimus, or actinomycin-D. In certain embodiments, the therapeutic agent includes a single anti-proliferative agent.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 depicts a lead and pulse generator in accordance with at least one embodiment.

FIG. 2A depicts a portion of a lead including a coating in accordance with at least one embodiment.

FIGS. 2B-2E depict a portion of a lead including a coating provided in accordance with various embodiments of the present invention.

FIG. 3 is a graph depicting the elution rates of clobetasol in porcine serum.

FIG. 4 is a graph showing the effect of a primer layer on the elution rate of clobetasol in porcine serum.

FIG. 5 is a graph demonstrating the effect of a topcoat layer on the elution rate of clobetasol in porcine serum.

FIG. 6 is a graph depicting the elution rates of sirolimus in porcine serum.

FIG. 7 is a graph depicting the elution rates of clobetasol in porcine serum from medical adhesive coated tubes.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

The present invention takes advantage of thin coatings of polymers and/or agents, such as therapeutic agents, applied to at least a portion of leads and/or electrodes. Thin coatings, instead of plugs and collars, reduce the polymer burden as well as allow for even distribution of agents, including high potency therapeutic agents, and/or polymers on leads and/or electrodes. Additionally, thin coatings allow for the creation of leads with smaller diameters (no longer necessary to accommodate the plug or collar). Thus, one embodiment provides for the combination of agents with downsized implantable devices. The coatings may also provide reduced acute and/or chronic pacing thresholds and/or increased sensing activity by the electrodes.

The term “lead” is used herein in its broadest sense and includes, but is not limited to, a stimulation lead, a sensing lead or a combination thereof. In one embodiment, the lead is adapted for active fixation. In another embodiment, the lead is adapted for passive fixation. In yet another embodiment, the lead is adapted for bipolar stimulation. In other embodiments, the lead is adapted for defibrillation and/or pacing/sensing. In one embodiment, the lead is tripolar or quadrupolar having a plurality of electrodes located on the lead body.

The lead may be adapted to deliver a variety of electrical stimulus therapies. According to some embodiments, the lead can be adapted to deliver an electrical stimulus therapy to the appropriate region of a patient's heart. In other embodiments, the lead can be a neurostimulation lead having reduced dimensions suitable for delivery into a patient's intracranial region. In yet another embodiment, the lead can be a cochlear implant lead.

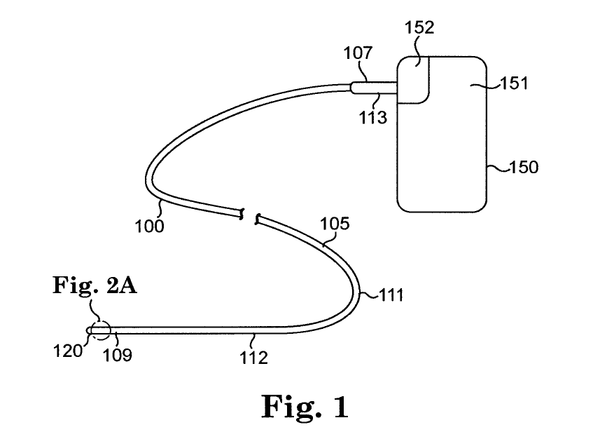

FIG. 1 shows a view of a lead 100 coupled to a pulse generator 150 according to one embodiment of the present invention. In one embodiment, lead 100 is adapted to deliver pacing energy to a heart. Some examples deliver defibrillation shocks to a heart. Pulse generator 150 can be implanted in a surgically-formed pocket in a patient's chest or other desired location. Pulse generator 150 generally includes electronic components to perform signal analysis, processing and control. Pulse generator 150 can include a power supply such as a battery, a capacitor and other components housed in a case or can 151. The device can include microprocessors to provide processing and evaluation to determine and deliver electrical shocks and pulses of different energy levels and timing for ventricular defibrillation, cardioversion and pacing to a heart in response to cardiac arrhythmia including fibrillation, tachycardia and bradycardia.

In one embodiment, lead 100 includes a lead body 105 extending from a proximal end 107 to a distal end 109 and having an intermediate portion 111. Lead 100 includes one or more conductors, such as coiled conductors or other conductors, to conduct energy from pulse generator 150 to an electrode 120, and also to receive signals from the heart. The lead further includes outer insulation 112 to insulate the conductor. The conductors are coupled to one or more electrodes, such as electrode 120. Lead terminal pins 113 are attached to pulse generator 150 at a header 152. The system can include a unipolar system with the case acting as an electrode or a bipolar system with a pulse between two distally located electrodes. In some examples, pulse generator can 151 can be used as an electrode. In some examples, a header electrode can be placed in or near the header 152 of can 151.

### Lead Coatings

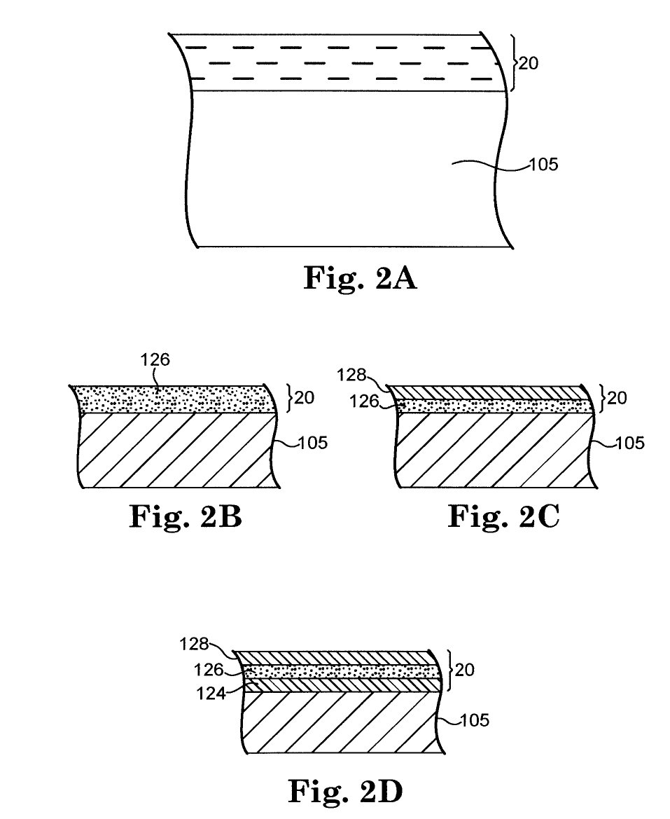

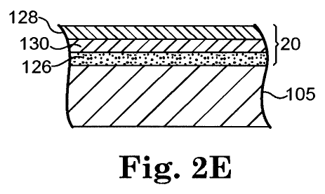

FIGS. 2A-2E depicts a coating 20 provided on at least a portion of the lead body 105 according to various embodiments of the present invention. According to one embodiment, the coating 20 is provided over one or more discrete sections located along an outer surface of the lead body 105. According to another embodiment, the coating 20 is provided over a majority (e.g. 80-95%) of the outer surface of the lead body 105 extending from the proximal end 107 to the distal end 109 of the lead body 105. According to yet another embodiment, the coating 20 is provided on the outer surface of the lead body 105 adjacent to one or more electrodes 120. According to still another embodiment, the coating 20 is provided over a distal portion of the lead body 105 such that it is adjacent to at least one electrode 120.

Generally, as shown in FIGS. 2B-2E, the coating 20 may include at least one of: a primer layer 124, a matrix polymer layer 126, and a topcoat layer 128. According to some embodiments, the matrix polymer layer 126 may include one or more agents admixed therein. The topcoat layer 128 may be a bio-beneficial topcoat and may include one or more agents admixed therein. A bio-beneficial topcoat layer is a layer that provides an anti-thrombogenic surface that may result in a moderate and controlled acute inflammatory response. The topcoat layer 128 may be provided over and adjacent to the matrix polymer layer 126 and/or the primer layer 124. In some embodiments, the coating 20 may include one or more agents 130 deposited on the lead body 105. The one or more agents 130 can elute through a layer such as the matrix polymer layer 126 and/or the topcoat layer 128.

According to one embodiment, as shown in FIG. 2B, the coating 20 includes at least one matrix polymer layer 126 provided over at least a portion of the lead 105. According to various embodiments, the matrix polymer layer 126 can include at least one polymer admixed with at least one therapeutic agent. In some embodiments, more than one matrix polymer layer 126 may be used to coat the lead body 105. Each matrix polymer layer 126 can include the same or different polymer and/or agent admixed therein.

According to another embodiment, as shown in FIG. 2C, the coating 20 includes at least one topcoat layer 128 provided over at least matrix polymer layer 126. The matrix polymer layer 126 and/or the topcoat layer 128 can include one or more agents admixed therein. According to some embodiments, one or more topcoat layers 128 may be provided in combination with one or more matrix polymer layers 126.

According to yet another embodiment, as shown in FIG. 2D, the coating 20 includes a primer layer 124, at least one matrix polymer layer 126 provided over the primer layer 124, and at least one topcoat layer 128 provided adjacent to the matrix polymer layer 128.

According to further exemplary embodiments, as shown in FIG. 2E, one or more agent layers 130 may be located adjacent to and in between, for example, a topcoat layer 128 and a matrix polymer layer 126.

### A. Primer Layer

According to one embodiment, the coating 20 includes a primer layer. The optional primer layer can be applied between the lead and another layer to improve the adhesion of the layer/coating 20 to the lead. The primer is applied to, for example, the outer surface of the lead body and/or electrode prior to application of another layer, such as the matrix polymer layer, optionally admixed with one or more agents, the topcoat layer, optionally admixed with one or more agent and/or the agent(s).

Primers include, but are not limited to, medical adhesives, acrylics and surface modification of the lead surface (e.g., silicone) with plasma, such as oxygen plasma (which modifies the surface of, for example, polymers (e.g., silicone), so that they can adhere with other materials, such as other layers within the coating 20 or adhesives). According to one embodiment, the primer layer 124 can include polybutylmethacrylate. According to other embodiments, the primer layer 124 may be a surface treatment layer. For example, the primer layer 124 can be created using a variety of surface treatment techniques including, but not limited to plasma etching, plasma deposition, chemical etching, vapor deposition, or other surface treatment techniques known to those of skill in the art.

### B. Matrix Polymer Layer

According to another embodiment the coating 20 includes at least one matrix polymer layer. According to further embodiments, the coating 20 can include more than one matrix polymer layer. Polymers for use in the matrix polymer layer include, but are not limited to, the following: Solef® (Solef® 21508 polymer); polyvinylidene-hexafluoropropylene or poly(VF2-co-HFP) from Solvay, Brussels, Belgium; Room-Temperature-Vulcanizing (RTV) silicone elastomers; silicone, polymers based on the structural unit R₂SiO, where R is an organic group; medical adhesives; cyanoacrylates; Rehau 1511; ethylene vinyl alcohol (ENAL; a thermoplastic polymer); polyethylene glycol (PEG); polyvinyl alcohol; polyvinyl propylene; hyaluronic acid; polyacrylamides; polycaprolactone, polylactide (PLA); polyglycolide (PGA); poly(lactide-co-glycolide) (PLGA); polyurethane; polymethylmethacrylates; polyethylene; polyvinylpyrrolidone; polyacrylic acid; poly(2-hydroxyethyl methacrylate); PHEMA polyacrylamide; polyethylene-co-vinyl acetate; polyanhydrides; polyorthoesters; polyimides; polyamides; polyanhydrides; polyetherketones; polyaryletherketones; polysiloxane urethanes; polyisobutylene copolymers; and copolymers and combinations thereof.

According to some embodiments, the matrix polymer layer can include one or more therapeutic agents admixed with at least one polymer, as described above. Any drug or bioactive agent which can serve as a useful therapeutic, prophylactic or even diagnostic function when released into a patient can be combined with a polymer to form the polymer matrix layer. Exemplary therapeutic agents include, but are not limited to, the following: an anti-inflammatory; anti-proliferative; anti-arrhythmic; anti-migratory; anti-neoplastic; antibiotic; anti-restenotic; anti-coagulation; anti-infectives; anti-oxidants; anti-macrophagic agents (e.g., bisphosphonates); anti-clotting (e.g., heparin, coumadin, aspirin); anti-thrombogenic; immunosuppressive agents; an agent that promotes healing, such as a steroid (e.g., a glucocorticosteriod) and/or re-endothelialization; and combinations thereof.

More specifically, the one or more therapeutic agents may include, but are not limited to, the following: paclitaxel; clobetasol; rapamycin; sirolimus; everolimus; tacrolimus; actinomycin-D; dexamethasone (e.g., dexamethasone, dexamethasone sodium phosphate or dexamethasone acetate); betamethasone; mometasone furoate; vitamin E; mycophenolic acid; cyclosporins; beclomethasone (e.g., beclomethasone dipropionate anhydrous); their derivatives, analogs, salts; and combinations thereof. Additionally, the one or more therapeutic agents may include bisphosphonates. Bisphosphonates inhibit macrophage-like action thereby limiting the local inflammatory response. According to yet other embodiments, the one or more therapeutic agents may include non-steroidal anti-inflammatory agents such as aspirin, ibuprofen, acetaminophen, and COX inhibitors (e.g., celecoxib and/or diclofenac).

According to some embodiments of the present invention, the matrix polymer layer includes a polymer combined with an anti-inflammatory agent. For example, the polymer can be combined with any one of dexamethasone, dexamethasone acetate, dexamethasone sodium phosphate, clobetasol, beclomethasone, or mometasone furoate. According to one embodiment, the matrix polymer layer includes silicone admixed with dexamethasone acetate. According to another embodiment, the matrix polymer layer includes a medical adhesive admixed with dexamethasone acetate. According to yet another embodiment, the matrix polymer layer includes a medical adhesive admixed with clobetasol. According to yet another embodiment, the matrix polymer layer includes poly(VF2-co-HFP) admixed with clobetasol. In still another embodiment, the matrix layer 126 includes polyurethane admixed with a COX inhibitor. According to other embodiments, the matrix layer 126 can include a polymer film including one or more hydrogel-like polymers (e.g., polyacrylamide, polyvinylpyrrolidone, PHEMA) containing one or more cyclosporine.

According to certain embodiments of the present invention, the matrix polymer layer 126 may include a polymer admixed with an anti-proliferative agent such as everolimus or paclitaxel.

According to still other embodiments of the present invention, a combination of an anti-proliferative (e.g., everolimus or paclitaxel) and an anti-inflammatory (e.g., dexamethasone, clobetasol or mometasone furoate) agent may be employed. In one embodiment, a combination of dexamethasone and everolimus is employed. In another embodiment, a combination of clobetasol and everolimus is employed. In yet another embodiment, a combination of dexamethasone and paclitaxel is employed. In another embodiment, a combination of clobetasol and paclitaxel is employed. In another embodiment, a combination of dexamethasone and sirolimus is employed. In one embodiment a combination of clobetasol and sirolimus is employed.

The therapeutic agent can be present in the polymer matrix layer in any effective amount. An “effective amount” generally means an amount which provides the desired local or systemic effect. For example, an effective dose is an amount sufficient to affect a beneficial or desired clinical result. The precise determination of what would be considered an effective dose may be based on factors individual to each patient, including their size and age.

The matrix polymer layer can be formed such that it includes an effective drug to polymer ratio (D:P). The drug to polymer ratio (D:P) can be selected for specific adhesion and release properties. The release rate of the drug from the polymer matrix can be manipulated through selection of an appropriate drug to polymer ratio to achieve the desired drug release profile. The drug to polymer ratio in the matrix polymer layer can be selected such that the drug release profile is immediate, short term, or sustained release. A matrix polymer layer having an immediate release profile releases the drug content within minutes to about an hour after implantation. A matrix polymer layer having a short term release profile more slowly liberates the content within days to weeks following implantation. Finally, a matrix polymer layer having a sustained release profile releases the content very slowly, with full release requiring months to years. According to one embodiment, the drug to polymer ratio in the matrix polymer layer can be selected such that it ranges from 1:20 to 5:20. According to another embodiment, the drug to polymer ratio in the matrix polymer layer can be selected such that it ranges from 1:20 to 1:1. Typically, a matrix polymer layer including a higher drug to polymer ratio will have a faster drug release profile. Additionally, the selection of the polymer included in the matrix can also affect the release rate of the drug.

### C. Topcoat Layer

According to yet other embodiments, the coating 20 includes a topcoat layer 128. According to some embodiments, the topcoat layer 128 may be provided over the polymer matrix layer 126 or another layer at discrete locations along the lead body 105. According to other embodiments, the topcoat layer 128 may be provided over a polymer matrix layer 126 or another layer from substantially the proximal end to the distal end of the lead body 105. The presence of a topcoat layer 128 in the coating 20 alters the concentration gradient of the therapeutic agent to be eluted from the matrix polymer layer 126. In some cases, the presence of a topcoat layer 128 over the matrix polymer layer 126 including the therapeutic agent, slows the release of the therapeutic agent from the coating 20 into the implant region.

According to various embodiments of the present invention, the topcoat layer 128 may be formed from the same or different polymer used to form the matrix polymer layer 126. Topcoat layers 128, such as bio-beneficial polymer topcoats, can be formed from compounds including, but not limited to, the following: phosphorylcholine (PC); polyvinylpyrrolidone (PVP); poly(vinyl alcohol) (PVA); hyaluronic acid (HA); and/or polyactive (a block copolymer composed of polyethylene oxide (PEO) and polybutylene terpthalate (PBT)). Other exemplary materials for forming the topcoat layer 128 include, but are not limited to, the following: Solef® (Solef® 21508 polymer); polyvinylidene-hexafluoropropylene or poly(VF2-co-HFP) from Solvay, Brussels, Belgium; Room-Temperature-Vulcanizing (RTV) silicone elastomers; silicone, polymers based on the structural unit R₂SiO, where R is an organic group; medical adhesives; cyanoacrylates; Rehau 1511; ethylene vinyl alcohol (E/VAL; a thermoplastic polymer); polyethylene glycol (PEG); polyvinyl propylene; polyacrylamides; polycaprolactone; polylactide (PLA); polyglycolide (PGA); poly(lactide-co-glycolide) (PLGA); polyurethane; polymethylmethacrylates; polyethylene; polyvinylpyrrolidone; polyacrylic acid; poly(2-hydroxyethyl methacrylate); PHEMA polyacrylamide; polyethylene-co-vinyl acetate; polyanhydrides; polyorthoesters; polyimides; polyamides; polyanhydrides; polyetherketones; polyaryletherketones; polysiloxane urethanes; polyisobutylene copolymers; and copolymers and combinations thereof.

In some embodiments, the topcoat layer 128 can be mixed with other components such as the materials used to form the matrix polymer layer 126 discussed above. In another embodiment, the topcoat layer 128 is applied on top of a matrix polymer layer 126 and/or agent layer 130. Like the matrix polymer layer 126, the topcoat layer 128 can include one or more therapeutic agents admixed with one or more topcoat components, described above. According to another embodiment, a topcoat material can be admixed within the matrix polymer layer 126.

According to further embodiments of the present invention, one or more topcoat layers 128 can be provided on the surface of an electrode 120. By coating the electrode 120 with a topcoat layer 128, the patient's immune system is exposed to an inert polymer and not the metal electrode 120. For example, it is believed that a phosphorycholine (solution in ethanol) layer functions as an anti-macrophage adhesion surface, while a sodium hyaluronate (HA) layer functions as an anti-platelet adhesion surface.

In one embodiment, the topcoat layer on at least a portion of the electrode 120 is bio-degradable (e.g., bio-dissolvable). Bio-degradable topcoat layers can be formed from such polymers including but not limited to polylactic acid, polyglycolic acid, and other biodegradeable polymers and substances known to those of skill in the art. In one embodiment, at least a portion of the lead 100 is coated with a bio-degradable topcoat layer. In another embodiment, at least a portion of the lead 100 is coated with a topcoat layer that is not biodegradable.

### D. Agents

One embodiment provides a drug eluting lead 100 which comprises at least one therapeutic agent 130. The agents may be used alone, in combinations of agents, admixed with a layer or applied on top of, underneath or between layers of the coating 20. In certain embodiments, the agent 130 is located between the other layers of the coating 20. For example, in some embodiments, the agent 130 may be located between two matrix polymer layers 126. In other embodiments, the agent 130 may be located between a topcoat layer 128 and a matrix polymer layer 126.

Exemplary therapeutic agents include, but are not limited to: anti-inflammatory, anti-proliferative, anti-arrhythmic, anti-migratory, anti-neoplastic, antibiotic, anti-restenotic, anti-coagulation, anti-infectives, anti-oxidants, anti-macrophagic agents (e.g. bisphosphonates), anti-clotting (e.g., heparin, coumadin, aspirin), anti-thrombogenic or immunosuppressive agent, or an agent that promotes healing, such as a steroid (e.g., a glucocorticosteriod), and/or re-endothelialization or combinations thereof. More specifically, the therapeutic agents may include, but are not limited to paclitaxel, clobetasol, rapamycin (sirolimus), everolimus, tacrolimus, actinomycin-D, dexamethasone (e.g., dexamethasone sodium phosphate or dexamethasone acetate), mometasone furoate, vitamin E, mycophenolic acid, cyclosporins, beclomethasone (e.g., beclomethasone dipropionate anhydrous), their derivatives, analogs, salts or combinations thereof. In essence, any drug or bioactive agent which can serve a useful therapeutic, prophylactic or even diagnostic function when released into a patient can be used.

In one embodiment, a combination of an anti-proliferative (e.g., everolimus or paclitaxel) and an anti-inflammatory (e.g., dexamethasone, clobetasol or mometasone furoate) agent may be employed. For example, in one embodiment, a combination of dexamethasone and everolimus is employed. In another embodiment, a combination of clobetasol and everolimus is employed. In yet another embodiment, a combination of dexamethasone and paclitaxel is employed. In another embodiment, a combination of clobetasol and paclitaxel is employed. In another embodiment, a combination of dexamethasone and sirolimus is employed. In one embodiment, a combination of clobetasol and sirolimus is employed. In other embodiments, a single therapeutic agent may be employed.

The therapeutic agent can be present in any effective amount. An “effective amount” generally means an amount which provides the desired local or systemic effect. For example, an effective dose is an amount sufficient to affect a beneficial or desired clinical result. The precise determination of what would be considered an effective dose may be based on factors individual to each patient, including their size and age. In one embodiment, the therapeutic agent is present in a concentration of less than about 100 μg/cm². For example, the agent may be present in a range of about 2 to about 10 μg/cm², about 10 to about 20 μg/cm², about 20 to about 30 μg/cm², about 30 to about 40 μg/cm², about 40 to about 50 μg/cm², about 50 to about 60 μg/cm², about 60 to about 70 μg/cm², about 70 to about 80 μg/cm², about 80 to about 90 μg/cm² and/or about 90 to about 100 μg/cm². The agents may also be present at a concentration of higher than about 100 μg/cm².

According to various embodiments of the present invention, a drug eluting lead can be delivered to a desired site within the patient's body. Once implanted, the therapeutic agent may elute from the surface of the implant and diffuse into the adjoining tissue. In this manner, the inflammatory process and/or other unwanted biological processes associated with implantation and the presence of the foreign object is suppressed (e.g., reduced inflammation and/or toxicity of inflammatory response). Additionally, the growth of non-excitable, connective tissue around the electrode (e.g., the capsule) is reduced (e.g., a reduction in fibrotic capsule thickness may be observed), and thus, the postoperative rise in the stimulation threshold lessens, a stable reduced threshold, both acute and chronic, is thereby provided. Additionally, the device and methods may prevent myocyte cell function impairment and/or necrosis around, near or on an electrode 120, which may further stabilize a reduced threshold.

In one embodiment, the therapeutic agent is available immediately after and/or during implantation (time of injury). In another embodiment, the therapeutic agent is available within a few days, such as about 1 to about 5 days. Following implantation, the agent has nearly completely eluted. In another embodiment, the therapeutic agent elutes in a couple of hours to several days to several weeks (e.g., in about 1 to about 5 weeks). The therapeutic agent may also be designed to have longer eluting times, such as several months. Additionally, the lead may be designed so that one therapeutic agent is released at the time of implantation (time of injury), while another therapeutic agent releases more slowly, for example, over the course of about several weeks to about a month or two from the time of implantation. In one embodiment, the two therapeutic agents may be the same or different therapeutic agents.

The release rate of the therapeutic agent can be controlled through the drug to polymer ratio in the one or more layers present in the coating provided on the lead body. According to one embodiment the drug to polymer ratio in the matrix polymer layer can be selected such that it ranges from 1:20 to 5:20. The number and layers included in the coating and the coating selected for each layer will also affect the release rate of the drug into the surrounding implantation site. Additionally, the inclusion of more than one therapeutic agent in a given layer may also affect the release rate of each therapeutic agent included therein.

### E. Method of Manufacture

In one embodiment, at least one agent, polymer and/or topcoat are admixed, for example, with a solvent to provide a solution or mixture. In one embodiment, the solvent does not interfere with the activity of the agent. Examples of such solvents include, but are not limited to, the following: water, alcohol, cyclohexanone, acetone, Freon®, xylenes, ethers, pentane, hexane, heptane, tetrahydrofuran (THF), dimethylformamide (DMF), dimethyl sulfoxide (DMSO), combinations thereof, and the like. The solution can be applied to at least a portion or all of a lead 100 and/or electrode 120 by, for example, spray coating. After the solvent in the solution is evaporated, a thin layer containing at least one agent, polymer and/or topcoat remains on the surface of the lead 100 and/or electrode 120. The process can be repeated as many times as desired to provide for multiple layers. Alternatively, the coating 20 can be applied to the lead 100 and/or electrode 120 by dip-coating. Brush-coating and RF magnetron physical vapor deposition sputtering process are alternative coating processes that may also be employed. The coating 20 may also be applied to the lead 100 using a combination of techniques including: spraying, dipping, sputtering and/or brushing.

In one embodiment, a coating 20 comprising one or more layers ranges from about submicron to about 10 microns in thickness, about 1 to about 50 microns in thickness or about 50 to about 100 microns in thickness. In another embodiment, the thickness of the coating 20 ranges from about 1 to about 5, about 5 to about 10 microns, about 10 to about 15, about 15 to about 20, about 20 to about 30, about 30 to about 40, about 40 to about 50, about 50 to about 60, about 60 to about 70, about 70 to about 80, about 80 to about 90, or about 90 to about 100. In one embodiment, one or more layers are distributed evenly across a distal portion of a lead 100 and/or electrode 120. In one embodiment, one or more layers are applied to the lead body 100 adjacent to the electrode 120. According to yet another embodiment, one or more layers are applied at discrete locations along the lead body 100.

A syringe coating apparatus may be used to apply the primer, the polymer matrix layer, with or without one or more agents admixed therein, the topcoat layer, with or without one or more agents admixed therein, and/or an agent to at least a portion of a lead and/or an electrode. For example, a syringe, typically a motorized syringe (filled with one or more agent, polymer and/or topcoat, or a mixture thereof in solution or as a mixture in solvent) mounted on a syringe pump (e.g., a positive displacement pump that can accurately meter fluid, the advancement of which is controlled by a motor, such as a step motor) can be connected to a hypodermic needle based nozzle assembly and can be used to apply one or more layers to at least a portion of a lead and/or electrode. The fluid dispensed from the needle can either be atomized to spray using pressured air (air inlet) on the nozzle or just droplets without using pressured air for coating at least a portion of the lead and/or electrode. The fluid can be dispensed at a predetermined rate. The lead can be rotated during this process so that all sides of the device are coated. A microscope can be attached to the apparatus to assist in visualizing the coating procedure. The samples may be mounted on the device under the microscope.

This process of spray coating allows for greater control of coating placement which thereby allows for more accurate placement so as to selectively coat one area of the lead and/or electrode without contaminating other areas of the lead and/or electrode with the spray solution/mixture. Other benefits of the spray coating method are decreased waste of coating solution/mixture and uniform coating on the device (e.g., along a lead body or on an electrode). A uniform thickness and precise quantity will lead to uniform and consistent eluting of agent from the coated device surface.

Additionally, coating of at least a portion of the lead 100 and/or the electrode 120 allows for therapeutic agent to be provided to the injured tissue over a larger region within a patient's body not restricted to the implant location. For example, a coating 20 including a matrix polymer layer 126 having a therapeutic agent can be provided at a location adjacent to the electrode 120 as well as at a location on the lead body 105 proximal to the electrode 120. Thus, the coating 20 can deliver a two-fold effect. The coating 20 or portion thereof provided adjacent the electrode 120 may assist in lower pacing thresholds at the site where the electro-stimulus therapy is delivered. The coating 20 or portion thereof provided at a more proximal location on the lead body 105 may provide an anti-inflammatory effect at a region where potential inflammation of bodily tissue may occur due to the presence of the lead body 105. Additionally, thin coatings and potent (chemically or medicinally effective) therapeutic agents provide for reduced polymer and therapeutic agent burden on the lead 100 and/or electrode 120, making it possible to reduce the lead 100 diameter.

Any combination of layers (primer, polymer matrix layer, topcoat layer) and/or agents is envisioned; additionally, the various components (primer layer 124, matrix polymer layer 126, topcoat layer 128, and/or agents 130) may be provided on the lead body 105. In one embodiment, the one or more layers and/or agent(s) are disposed over at least a portion of the lead body 105 adjacent to the at least one electrode 120. For example, in one embodiment, the agent(s) and/or layers(s) are applied directly to at least a portion of the lead body 105 and/or electrode 120. In another embodiment, at least a portion of the lead body 105 and/or electrode 120 is coated with a primer layer. In another embodiment, at least a portion of the lead body 105 is coated with primer layer 124 and/or a matrix polymer layer 126. In another embodiment, at least a portion of the lead body 105 is coated with primer layer 124, matrix polymer layer 126 and/or a topcoat layer 128. In another embodiment, at least a portion of the lead body 105 is coated with matrix polymer layer 126. In another embodiment, at least a portion of the lead body 105 is coated with a matrix polymer layer 126 and/or a topcoat layer 128. In another embodiment, at least a portion of the lead body 105 and/or electrode 120 are coated with topcoat layer 128. In another embodiment, at least a portion of the lead 100 and/or electrode 120 are coated with agent (e.g., therapeutic agent or drug).

In one embodiment, one or more agents 130 are applied directly onto at least a portion of the lead 100 and/or the electrode 120. In another embodiment, one or more agents 130 are applied on top of a primer layer 124, a matrix polymer layer 126, and/or a topcoat layer 128. In another embodiment, one or more agents 130 are admixed with the matrix polymer layer 126 and/or the topcoat layer 128 (e.g., prior to application of the layer). In another embodiment, one or more agents 130 are applied between two or more layers of matrix polymer layer 126 and/or two or more layers of the topcoat 126. The agents 130 admixed in the layers and/or applied on top of or between the layers can be the same or different. For example, in one embodiment, an agent admixed with the polymer matrix layer is different from an agent admixed in the topcoat layer.

One embodiment provides a matrix polymer layer 126 applied alone to at least a portion of the lead 100, applied after a primer layer 124, applied after an agent 130, and/or admixed with one or more agents 130, and/or followed by another polymer matrix layer 126 and/or a topcoat layer 128 or agent 130. Another embodiment provides a bio-beneficial topcoat over one or a mixture of anti-inflammatory and anti-proliferative agents, including dexamethasone, such as dexamethasone acetate, clobetasol and everolimus in a polymer matrix. Another embodiment provides a lead body 105 comprising a bio-beneficial polymer topcoat 128 over a drug eluting polymer matrix layer 126 comprising clobetasol and/or everolimus in Solef®. Such a combination will give an anti-thrombogenic surface and will result in moderate and controlled acute inflammatory response.

In one embodiment, a topcoat layer 128 is admixed with one or more agents 130. Alternatively, the agent 130 is applied before or after the topcoat 128 or in between two layers of topcoat 128. The topcoat layer 128 can be applied directly to at least a portion of the lead body 105 and/or electrode 120. A topcoat layer 128 can also be applied to the matrix polymer layer 126, admixed with the matrix polymer layer 126, or applied over another topcoat layer 126.

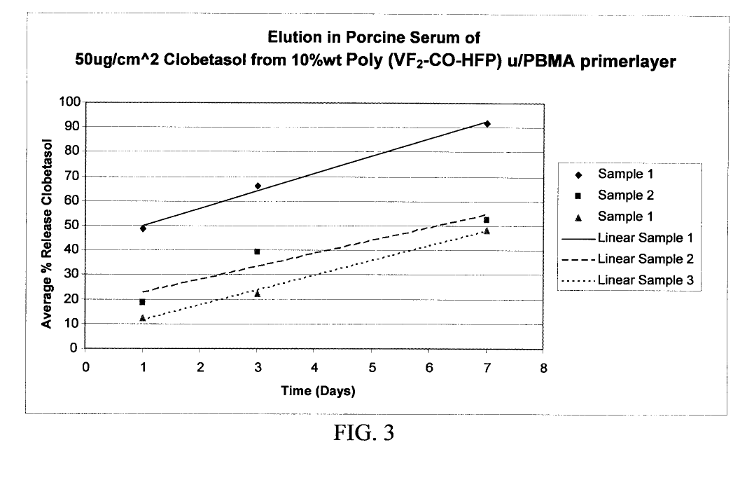

In addition to the agent 130 and/or layers/coatings 20 being deposited on the surface of at least a portion of the electrode 120, the agent 130 may be deposited within interstices of a porous electrode (e.g., a porous platinum electrode) and/or other types of depressions (e.g., channels, grooves, bore holes) of the electrode 120. As a result of the addition of structure to the electrode 120, an increased amount of agent 130, primer layer 124, matrix polymer layer 126, and/or topcoat layer 128 may be deposited. The primer layer 124, matrix polymer layer 126, topcoat layer 128 and/or agent 130 may be applied into channels via an inkjet device or the syringe/needle apparatus depicted in FIG. 3 or any other methods described herein.

In one embodiment, the agent 130, primer layer 124, matrix polymer layer 126 and/or topcoat layer 128 are applied to at least a portion of an electrode 120 which contacts tissue when implanted. In one embodiment, the coatings 20 and/or agent(s) do not impede the function of the lead 100 and/or electrode 120 (e.g., the electrode 120 can pace through the coating 20 and/or agent(s)). In one embodiment, the agent, primer, polymer matrix and/or topcoat are applied to at least a portion of a lead 100 and to at least a portion of an electrode 120.

Additionally, the primer layer 124, matrix polymer layer 126, topcoat layer 128 and/or agent 130 can be combined, cast into films and mounted on a lead body 105 as a drug collar or formed into a polymer plug. For example, an electrode, such as a Fineline electrode tip (a cathode comprised of crenulated dome having a surface of polished platinum, platinum black, platinum/iridium, iridium oxide, titanium nitride, or other suitable electrode material), can be formulated so as to comprise a polymer plug of, for example, one or more agents and at least one polymer or topcoat. In one embodiment, the agents comprise a steroid and everolimus. In another embodiment, the therapeutic agent comprises everolimus. In one embodiment, the agent and polymer are admixed; in another embodiment, they are layered. The plug can be pre-made and inserted in the electrode or can be deposited in the space using syringe technology.

In one embodiment, dexamethasone (e.g., dexamethasone sodium phosphate or dexamethasone acetate) and an anti-proliferative agent, such as everolimus, is delivered through a silicone collar and/or plug. In another embodiment, sodium hyaluronate (HA) is used as a drug delivery vehicle for anti-inflammatory and/or anti-proliferative agents in a plug and/or collar. In one embodiment, at least a portion of a lead helix, lead and/or electrode is coated with a mixture of HA and phosphorylcholine (PC) or a layer of PC followed by a layer of HA. Another embodiment provides a plug comprising a mixture of HA/PC/everolimus/dexamethasone acetate. Another embodiment provides a collar comprising a mixture of HA/PC/everolimus/dexamethasone acetate coated with layers of HA and PC.

As used herein, a coating associated with an electrode includes but is not limited to a layer on the surface of the electrode; components described herein may be within interstices of a porous electrode (e.g., a porous platinum electrode) and/or other types of depressions (e.g., channels, grooves, bore holes) of the electrode, and drug plugs.

The coating 20, which comprises one or more layers, is useful on any medical lead. For example, any medical implantable lead including, but not limited to, right-sided and left-sided cardiac leads, positive fixation leads where therapeutic agent is positioned at the fixation mechanism, positive fixation leads where therapeutic agent is positioned at the fixation mechanism that includes an electrode helix, epicardial leads that are sized for implantation through catheter delivery systems, downsized leads where coatings 20 are an option for positioning controlled release therapeutic agent delivery technology, neuro-stimulation leads requiring precise placement of electrode/therapeutic agent releasing components, miniaturized electrodes where coatings 20 can mask to produce high impedance and release agents, and miniaturized leads where a plurality of electrodes can be produced at specific locations by coating/masking.

### EXAMPLES

### Example 1

### Sample Preparation

The sample substrates were fabricated from stainless steel bar stock. Each sample was a 2.5″ long and 0.094″ diameter pin including a groove. The groove dimensions simulate the dimensions of a drug collar on a lead body.

Drug and polymer solutions for each test sample were prepared using the following procedures. First, a stock solution of poly(VF2-co-HFP) in acetone (10% w/w) was prepared. The stock solution was then used to prepare the final drug and polymer solution.

The requisite amount of poly(VF2-co-HFP) polymer was weighed in a vial to create a final polymer concentration of 10% (w/w) in solvent. Acetone was added to the vial with poly(VF2-co-HFP) polymer to create a solution with final acetone-to-cyclohexanone concentration of 80% acetone. The solution was stirred until all of the polymer was dissolved.

PBMA solution was prepared to create final solution of 0.8% (w/w) PBMA in 20% (w/w) cyclohexanone in acetone. The solution was stirred over low heat to affect dissolution of polymer.

Varying drug solutions were prepared for coatings including a final solution of 0.2-10% (w/w) poly(VF2-co-HFP) in 20% (w/w) cyclohexanone in acetone. Solutions including the matrix polymer were prepared by diluting poly(VF2-co-HFP) stock solution in 20% (w/w) cyclohexanone in acetone. Clobetasol was added to the matrix polymer solution in drug-to-polymer ratios (D:P) of 1:1 to 1:20. Drug was added to the solution to create a final theoretical drug dose of 50 μg/cm² of clobetasol. The solution was stirred until all drug is dissolved. Topcoats including only the matrix polymer, poly(VF2-co-HFP) were prepared in a manner identical to the drug solution minus the addition of drug.

### Samples

TABLE 1
50 μg/cm² Clobetasol in 10% wt poly(VF2-co-HFP)

| Sample | Drug to Polymer Ratio |
|---|---|
| 1 | 1:5 |
| 2 | 1:10 |
| 3 | 1:20 |

### Coating Apparatus and Coating Procedure

A syringe coating apparatus, such as previously described, was used to coat polymer and drug solution onto the sample pins.

In the samples including a primer layer, the primer layer was applied to the sample substrate first and the matrix polymer layer, including clobetasol, was applied over the primer layer. The primer layer and matrix polymer layer were applied on the sample substrate using the procedure described below.

Sample pins were sonicated in acetone prior to coating. The pins were mounted under the microscope of the coating fixture and the pin groove was located. A 10 μL syringe was loaded with the appropriate solution: primer, drug, or top coat, and purged. The pin was rotated as the syringe pump dispensed the solution at a pre-set rate. The coated sample was then mounted onto a sample holder and placed in a 50° C. oven for 2 hours to dry.

### Drug Elution Testing Procedure

Porcine serum was used as the elution medium. The elution time points were 1, 3, and 7 days. The pin samples were each dipped into 10 ml of porcine serum with 0.1% sodium azide as a stabilizer. The pins were moved up and down (dipped) in the porcine serum at 40 dips per min. The release rate tester bath temperature was maintained at 37° C. The porcine serum was changed every 36 hours for the three day sample, and for the seven day sample, the porcine serum was changed in 24, 48, and 72 hours. The pins were subjected to total drug content testing. The eluted drug was calculated by subtracting the drug left in the coating from the total drug content obtained from samples (mean value of the three samples) from the same group that were not subjected to the elution testing. Three samples per time point per group were used for the drug elution testing.

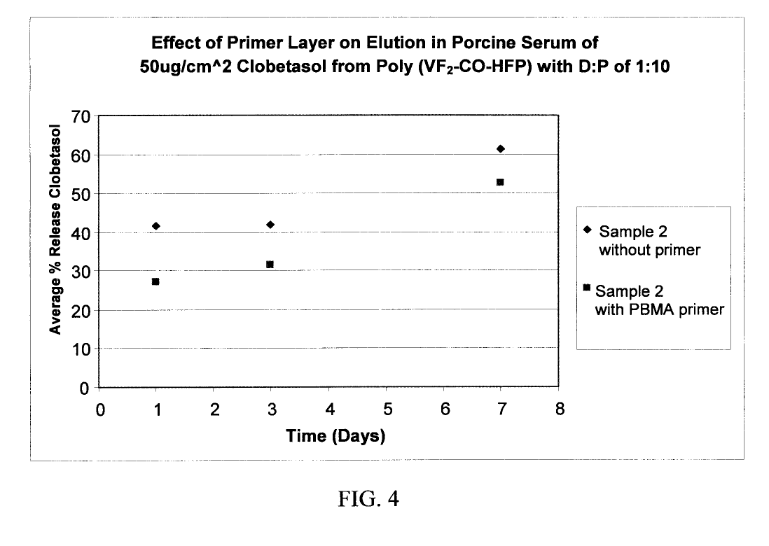

The drug elution testing results for sample nos. 1-4 are graphically represented in FIGS. 3 and 4. FIG. 3 shows the different drug elution rates for each D:P ratio tested. FIG. 4 shows the drug elution rate for sample no. 2 prepared with and without a polybutyl methylmethacrylate (PBMA) primer layer present on the sample substrate.

### Example 2

A drug solution including 5 μg/cm² clobetasol in 10% wt poly(VF2-co-HFP) having a D:P of 1:20 was prepared according to the procedure described above. To prepare the test samples, a PBMA primer layer was first coated onto the sample substrate pin according to the method described above. A layer including the drug solution was applied over the primer layer. To prepare the next sample, a topcoat layer including 10% wt poly(VF2-co-HFP) was applied followed by the drug solution layer.

TABLE 2

| Sample No. | Poly(VF2-co-HFP) Topcoat |
|---|---|
| 4 | No |
| 5 | Yes |

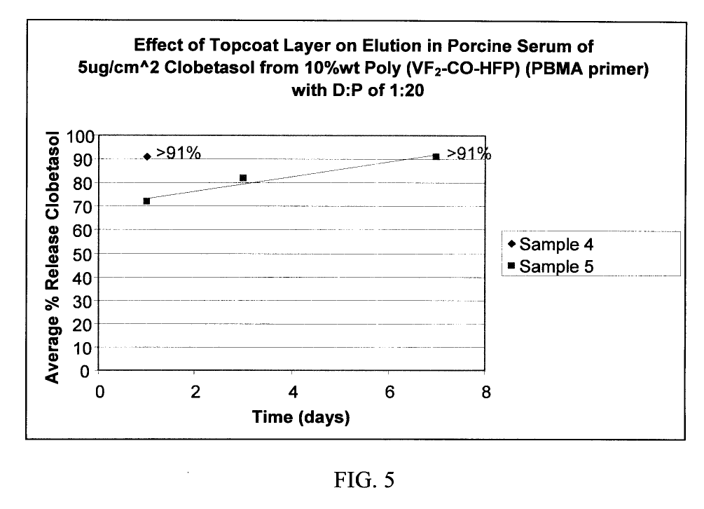

Drug elution testing on samples 4 and 5 was conducted according to the procedure described above. The results are graphically represented in FIG. 5. As demonstrated in FIG. 5, the presence of a topcoat layer decreased the elution rate of the drug from the sample.

### Example 3

A first drug solution including 100 μg/cm² sirolimus in 10% wt poly(VF2-co-HFP) having a D:P of 1:2 was prepared according to the procedure described above. A second drug solution including 300 μg/cm² sirolimus in 10% wt poly(VF2-co-HFP) having a D:P of 1:1 was also prepared. Test samples were prepared using both of the first and second drug solutions. Test samples were prepared with and without a primer layer. To prepare the test samples having a primer layer, a PBMA primer layer was first coated onto the sample substrate pin according to the method described above. A layer including the drug solution was applied over the primer layer.

TABLE 3

| Sample No. | Concentration | Drug to Polymer Ratio | Primer Layer |
|---|---|---|---|
| 6 | 100 μg/cm² | 1:2 | No |
| 7 | 100 μg/cm² | 1:2 | Yes |
| 8 | 300 μg/cm² | 1:1 | No |
| 9 | 300 μg/cm² | 1:1 | Yes |

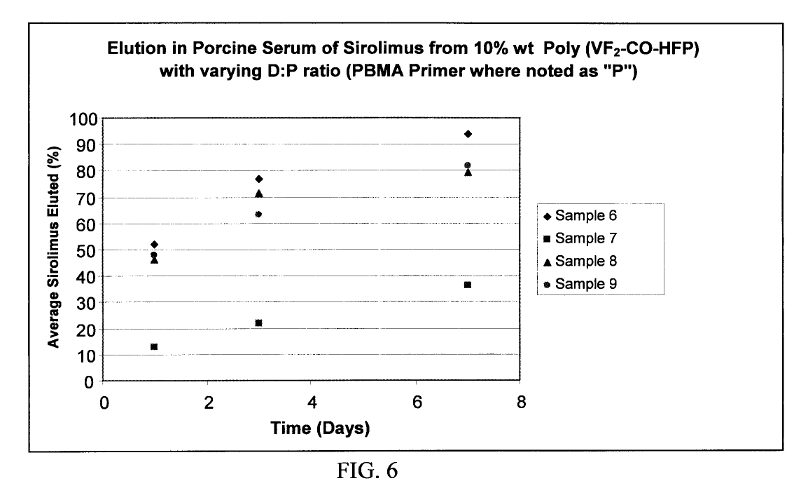

Drug elution testing was conducted according to the procedure described above. The testing results are graphically represented in FIG. 6. FIG. 6 graphically demonstrates the effect of both the drug to polymer ratio and the presence of a primer layer for each of the samples tested.

### Example 4

### Substrate Sample Description

The sample was prepared on pieces of cut silicone tubing.

### Drug Solution Preparation

A medical adhesive (MA) solution was prepared for a coating having a final solution of 20% (w/w) MA in 20% (w/w) cyclohexanone in xylenes. Drug was added to the MA solution to create a final theoretical drug dose of 200 μg/cm² of clobetasol. The solution was stirred until all drug was dissolved. Various drug solutions were prepared having a drug-to-polymer ratio (D:P) ranging from 1:2 to 1:20.

### Coating Apparatus and Coating Procedure

A syringe coating apparatus, previously described above, was used to coat polymer and drug solution on the samples.

The samples were mounted under the microscope of the coating apparatus. A 10 μl syringe was loaded with the appropriate solution, drug or top coat, and purged. The sample was rotated as the syringe pump dispensed the solution at a set rate. The coated sample was mounted onto a sample holder and placed in a 50° C. oven for 6 hours followed by 24 hours at room temperature to dry.

### Drug Elution Testing Procedures

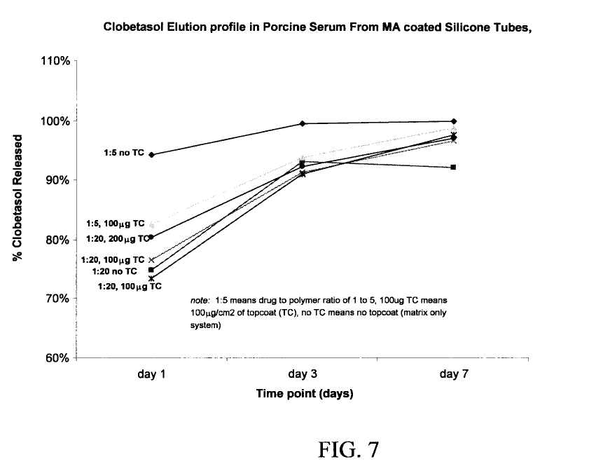

Porcine serum was used as the elution medium. The elution time points were 1, 3, and 7 days. The samples were each dipped into 10 ml of porcine serum with 0.1% sodium azide as a stabilizer. The samples were moved up and down (dipped) in the porcine serum at 40 dips per min. The release rate tester bath temperature was maintained at 37° C. The porcine serum was changed every 36 hours for the three day sample, and for the seven day sample, the porcine serum was changed in 24, 48, and 72 hours. The samples were subjected to total drug content testing. The eluted drug was calculated by subtracting the drug left in the coating from the total drug content obtained from samples (mean value of the three samples) from the same group that were not subjected to the elution testing. Three samples per time point per group were used for the drug elution testing. The results for the different samples that were tested are graphically represented in FIG. 7.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical electrical lead comprising:
a lead body having an outer surface extending from a proximal end adapted to be connected to a pulse generator to a distal end;
at least one conductor operatively connected to the pulse generator extending within the lead body;
at least one electrode located on the lead body operatively connected to the at least one conductor; and
a drug eluting coating disposed over at least a portion of the outer surface of the lead body adjacent to the at least one electrode, the drug eluting coating including at least one matrix polymer layer comprising a polymer admixed with a therapeutic agent including an anti-inflammatory agent.

2. The medical electrical lead according to claim 1, wherein the anti-inflammatory agent is selected from the group consisting of dexamethasone, dexamethasone sodium phosphate, dexamethasone acetate, betamethasone; beclomethasone, clobetasol, and mometasone furoate.

3. The medical electrical lead according to claim 1, wherein the anti-inflammatory agent comprises dexamethasone, dexamethasone sodium phosphate, or dexamethasone acetate.

4. The medical electrical lead according to claim 1, wherein the polymer comprises a medical adhesive, silicone rubber, or poly(VF2-co-HFP).

5. The medical electrical lead according to claim 1, wherein the drug eluting coating further comprises a primer layer deposited on the outer surface of the lead body.

6. a topcoat layer deposited adjacent the matrix polymer layer, or both.

7. The medical electrical lead according to claim 1, wherein the drug eluting coating has a drug to polymer ratio (D:P) ranging from about 1:20 to about 1:1.

8. The medical electrical lead according to claim 1, wherein the drug eluting coating is about 5 to about 10 microns thick.

9. The medical electrical lead according to claim 1, wherein the matrix polymer layer consists essentially of silicone rubber and dexamethasone.

10. The medical electrical lead according to claim 1, wherein the matrix polymer layer consists essentially of a medical adhesive and clobetasol or dexamethasone acetate.

11. The medical electrical lead according to claim 1, wherein the matrix polymer layer consists essentially of poly(VF2-co-HFP) and clobetasol.

12. The medical electrical lead according to claim 1, wherein the drug eluting coating comprises a polymer admixed with a therapeutic agent consisting of a single anti-inflammatory agent.

13. A medical electrical lead comprising:
a lead body having an outer surface extending from a proximal end adapted to be connected to a pulse generator to a distal end;
at least one conductor operatively connected to the pulse generator extending within the lead body;
at least one electrode located on the lead body operatively connected to the at least one conductor; and
a drug eluting coating disposed over at least a portion of the outer surface of the lead body adjacent to the at least one electrode, the drug eluting coating including at least one matrix polymer layer comprising a polymer admixed with a therapeutic agent including an anti-proliferative agent.

14. The medical electrical lead according to claim 13, wherein the anti-proliferative agent comprises paclitaxel, sirolimus, everolimus, tacrolimus, or actinomycin-D.

15. The medical electrical lead according to claim 13, wherein the matrix polymer layer consists essentially of poly(VF2-co-HFP) and sirolimus.

16. The medical electrical lead according to claim 13, wherein the matrix polymer layer consists essentially of poly(VF2-co-HFP) and everolimus.

17. The medical electrical lead according to claim 13, wherein the matrix polymer layer consists essentially of poly(VF2-co-HFP) and paclitaxel.

18. The medical electrical lead according to claim 13, wherein the drug eluting coating comprises a polymer admixed with a therapeutic agent consisting of a single anti-proliferative agent.

19. The medical electrical lead according to claim 18, wherein the single anti-proliferative agent is sirolimus.
